(19) United States
(12) Patent Application Publication
(10) Pub. No.: US 2025/0213524 A1
(43) Pub. Date: Jul. 3, 2025

(54) COMPOSITIONS FOR USE

(71) Applicant: Amazentis SA, Lausanne (CH)

(72) Inventors: Christopher L. Rinsch, Lausanne (CH); Anurag Singh, Lausanne (CH); Davide D'Amico, Renens (CH)

(21) Appl. No.: 18/990,754

(22) Filed: Dec. 20, 2024

(30) Foreign Application Priority Data
Dec. 22, 2023 (GB) 2319994.6

(51) Int. Cl. A61K 31/37; A61B 17/435; A61K 45/06; A61P 15/08

(52) U.S. Cl. CPC A61K 31/37; A61B 17/435; A61K 45/06; A61P 15/08

(57) ABSTRACT

The invention provides a compound of formula (I), or salt thereof,

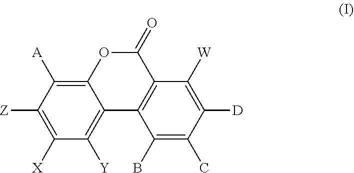

wherein:
A, B, C, D, W, X, Y and Z are each independently selected from H and OH; for use in a method of enhancing or improving fertility in a male or female subject, for example, a human male subject. The invention also provides a method of enhancing or improving fertility in a subject (for example a male subject) comprising administering a compound of formula (I) to the subject, and compositions that find use in such methods.

### RELATED APPLICATION

This application claims the benefit of priority to UK Patent Application No. 2319994.6, filed Dec. 22, 2023.

### BACKGROUND

Infertility is a condition generally characterized by a couple not being able to become pregnant after a year of trying. Infertility is fairly common in the U. S. and affects thousands of Americans every year. After one year of having unprotected sex, an average of 15 percent of couples are unable to get pregnant.

Generally, it is said that the following conditions are necessary for establishment of pregnancy.
(1) a certain number or more of spermatozoa with sufficient motor ability and fertilization ability are present in ejaculate semen,
(2) that follicles and eggs develop and mature normally, eggs with fertilizing ability are ovulated,
(3) that the morphology and function of the fallopian tube is maintained normally,
(4) The shape and function of the uterus are normal for implantation and maintenance of the embryo

Approximately 15% of human couples are infertile, of which male infertility accounts for 40-50%, and an estimated 7% of men experience problems in conceiving a child because of sperm defects. Overall fertility is decreasing worldwide, with the greatest effects in Western countries. In most cases the causes are unknown, although environmental factors are suspected. In almost half of infertile men, oligozoospermia or azoospermia is present despite unimpaired reproductive hormone secretion and a lack of other known determinants of infertility. Assisted conception including in vitro fertilization (IVF) and intra-cytoplasmic sperm injection is expensive, invasive and unreliable, and may cause epigenetic changes.

Fertility is also closely related to age in both women and men. The age of peak fertility for both genders is from their late teens to early thirties. After this age, there is a decline in fertility, to the point where it's very unusual for a woman to get pregnant using her own eggs after the age of 45 and it is much less likely (although by no means impossible) for a man in his fifties and beyond to father a child. However, many couples wait until they are older to start a family which may be due to focusing on careers or getting married later in life. Given the relationship between fertility and age, such couples will have greater difficulty in conceiving children.

Accordingly, there is a great need for new compositions and methods for the treatment and prevention of infertility for both couples in general and for older couples.

### SUMMARY

The present invention relates to the use of compositions for enhancing fertility in both male subjects and female subjects, including both animal and human male and female subjects, comprising one or more urolithins, for example, urolithin A. Compositions may be used as a supplement, for example, an oral supplement, in such male and female subjects or may be used in-vitro, for example, in in-vitro fertilization. The invention also relates to methods of enhancing fertility in male and female subjects comprising the step of administering said compositions.

Surprisingly, we have found that urolithins, for example, urolithin A, can be used in methods of enhancing fertility in both male and female subjects, and can be used to enhance methods of in-vitro fertilization. Such methods, have utility in both human subjects, and in animals, such as horses, sheep, bovines, pigs, etc.

According to an embodiment of the invention, a compound of formula (I), or salt thereof,

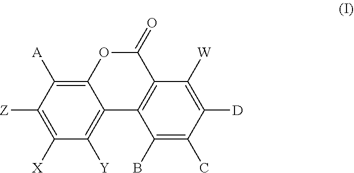

wherein:
A, B, C, D, W, X, Y and Z are each independently selected from H and OH;
for use in a method of enhancing or improving fertility in a male subject.

### BRIEF DESCRIPTION OF THE FIGURE

FIG. 1: A graph showing ovarian reserve decline with aging.

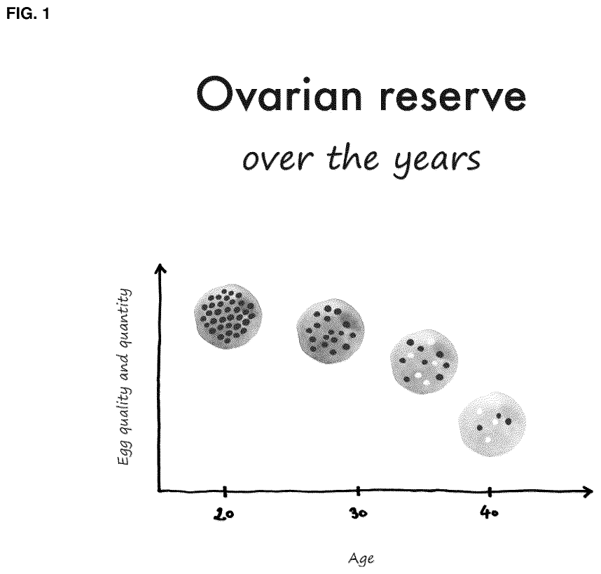

### DETAILED DESCRIPTION

According to an embodiment of the invention, disclosed is a compound of formula (I), or salt thereof,

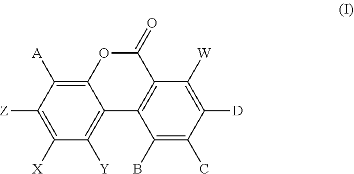

wherein:
A, B, C, D, W, X, Y and Z are each independently selected from H and OH;
for use in a method of enhancing or improving fertility in a male subject.

In one, the subject is a human male. In a further embodiment the subject is a male animal.

In one embodiment, the enhancement or improvement in male fertility is such that there is an increase of 10%, about 20%, about 30%, about 40%, about 50%, about 60%, about 70%, about 80%, about 90% or about 100% increase in the pregnancy rate.

In a further embodiment of the invention the enhancement or improvement in male fertility leads to an increase in pregnancy success rate.

Both men and women experience a decline in reproductive function with aging. Methods of increasing fertility of the invention can lead to an extending of reproductive function in both men and women. Therefore, according to a further embodiment of the invention, there is provided a compound of formula (I) for use in a method of extending reproductive longevity in a subject. In one embodiment the extension of reproductive longevity is in a male subject. In a further embodiment, the extension of reproductive longevity is in a female subject.

In a further embodiment, the enhancement or improvement in fertility is in a male subject, and comprises, one of more of the following:
a) an improvement in sperm quality;
b) an increase in sperm quantity;
c) an improvement in sperm motility;
d) an increase in rapid progressive motile sperm quantity;
e) an increase in sperm beat cross frequency;
f) an improvement in sperm maturation;
g) an improvement in sperm vitality;
h) an improvement in sperm function; and/or
i) an improvement in sperm health.

In a further embodiment, the enhancement or improvement in fertility comprises an increase in sperm quality and/or sperm motility.

In a further embodiment, the enhancement or improvement in fertility is in a male subject, and comprises, one of more of the following:
(a) An enhancement of sperm mitochondrial function;
(b) An increase in sperm mitochondrial mass, and or
(c) An increase in the quantity of mitochondria in sperm.

In a further embodiment, there is provided a compound of formula (I), for example, urolithin A, for use in a method for the treatment of prophylaxis of a disease, condition or disorder causing male infertility. In one embodiment, the disease, condition or disorder is selected from oligozoospermia, azoospermia, teratospermia and varicocele, for example, varicocele.

In a further embodiment, there is provided a compound of formula (I), for example, urolithin A, for use in a method for improving sperm function during aging.

In a further embodiment, there is provided a compound of formula (I), for example, urolithin A, for use in a method for reducing decline in sperm function during aging.

In a further embodiment, there is provided a compound of formula (I), for example, urolithin A, for use in a method for reversing decline in sperm function during aging.

In a further embodiment, there is provided a compound of formula (I), for example, urolithin A, for use in a method for enhancing sperm quality, for example sperm quality during aging. Sperm quality is defined as the percentage of functioning sperm in a sperm population.

In a further embodiment, the enhancement or improvement in fertility is in a female subject, and comprises, one of more of the following:
a) an increase in the quality or quantity of eggs;
b) a slowing oocyte aging;
c) an increasing in oocyte mitochondrial mass; and/or
d) preventing a decrease in oocyte mitochondrial mass.

In a further embodiment of the invention, there is provided a compound of formula (I), or salt thereof, as defined herein, for use in maintaining or enhancing ovarian health, in a female subject, for example, a human female subject.

Maintaining or enhancing ovarian health may comprise the treatment of a disease associated with the ovary. Therefore, in a further embodiment of the invention there is provided the use of a compound of formula (I) for maintaining or enhancing ovarian health wherein the use comprises the treatment of a disease, disorder or condition associated with the ovary.

In a further embodiment, the disease, disorder or condition associated with the ovary is selected from: polycystic ovary syndrome and pre-eclampsia.

In a further embodiment, the enhancing of ovarian health comprises maintaining or enhancing ovarian reserve in a female subject, for example, a human female subject.

In a further embodiment, the invention provides a method of enhancing or improving fertility in a subject (for example a male subject) comprising administering a compound of formula (I), for example, urolithin A, to the subject

In a further embodiment of the invention, there is provided a compound of formula (I), for reducing, preventing, or delaying perimenopause, menopause, or a symptom thereof in a female subject.

In a further embodiment, there is provided a compound of formula (I), for example, urolithin A, for use in a method for the treatment of prophylaxis of a disease, condition or disorder causing female infertility.

In a further embodiment, there is provided a compound of formula (I), for example, urolithin A, for use in a method of reducing decline in female fertility during aging.

In a further embodiment, there is provided a compound of formula (I), for example, urolithin A, for use in a method of reducing decline in egg function during aging.

In a further embodiment, there is provided a compound of formula (I), for example, urolithin A, for use in a method of improving fertilized egg implantation.

In a further embodiment, there is provided a compound of formula (I), for example, urolithin A, for use in a method of improving fertilized egg fertilisation.

In addition to administering compounds of formula (I) to human male and/or human female subjects, compounds of formula (I) also find utility in methods of assisted reproduction, such as in-vitro fertilization. This includes in methods of sourcing eggs from a female, for example, a human female, increasing the chances of successful fertilization in-vitro, culturing embryos in-vitro, prior to implantation into the female and when transferring the embryos to the human female.

Therefore, according to a further embodiment of the invention, there is provided a compound of formula (I) for use in a method of egg harvesting from a human female.

According to a further embodiment of the invention, there is provided a compound of formula (I) for use in a method of human in-vitro fertilization. In one embodiment an egg or eggs are pretreated with a compound of formula (I), for example, urolithin A, prior to in-vitro fertilization. In a further embodiment, in-vitro fertilization is conducted in the presence of a compound of formula (I), for example, urolithin A. In a further embodiment, eggs are pretreated with a compound of formula (I), for example, urolithin A, prior to in-vitro fertilization and in-vitro fertilization is conducted in the presence of a compound of formula (I), for example, urolithin A.

According to a further embodiment of the invention, there is provided a compound of formula (I) for use in a method of improving egg fertilization rate or improving embryo development during a method of human in-vitro fertilization.

According to a further embodiment of the invention, there is provided a compound of formula (I) for use in a method for delaying aging of an egg cultured in-vitro. In such a use, the compound of formula (I) may be added to the culture medium or injected into directly into the egg.

According to a further embodiment of the invention, there is provided a compound of formula (I) for use in a method of in-vitro fertilization wherein the compound of formula (I) is administered to the subject after the fertilized egg or embryo has been transferred to the female subject.

### Culture Media of the Invention:

In addition to oral administration to male or female subjects, compounds of formula (I) may be added to cell culture media, for example, cell culture media for use in in-vitro fertilization or other assisted reproduction methods,

According to a further embodiment of the invention, there is provided a cell culture medium which comprises a compound of formula (I), for example, a cell culture medium for use in the culturing of cells being used in a process of in vitro fertilization. In one embodiment the cell culture medium comprises about 1 μM to about 50 μM of a compound of formula (I).

According to a further embodiment of the invention, there is provided a method of culturing a cell, for example, an egg, in vitro comprising growing or maintaining the cell in cell culture medium which includes a compound of formula (I), for example, urolithin A.

According to a further embodiment of the invention, there is provided a cell culture medium which comprises a compound of formula (I), as described herein, wherein the medium comprises one of more additional components.

The skilled person would be familiar with cell culture media, suitable for in-vitro fertilization, for example, these can be found in Gruber & Klein (2011) J Turkish-German Gynecol Assoc 12:110-117.

### Urolithins

Urolithins are metabolites produced by the action of mammalian, including human, gut microbiota on ellagitannins and ellagic acid. Ellagitannins and ellagic acid are compounds commonly found in foods such as pomegranates, nuts and berries.

Ellagitannins are minimally absorbed in the gut themselves. Urolithins are a class of compounds with the representative structure (I) shown above. The structures of some particularly common urolithins are described in Table 1 below, with reference to structure (I).

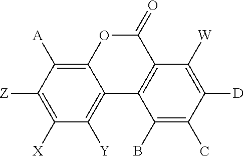

| | Substituent of structure (I) | | | | | |
|---|---|---|---|---|---|---|
| | A | B | C | D | W, X and Y | Z |
| Urolithin A | H | H | H | OH | H | OH |
| Urolithin B | H | H | H | H | H | OH |
| Urolithin C | H | H | OH | OH | H | OH |
| Urolithin D | OH | H | OH | OH | H | OH |
| Urolithin E | OH | OH | H | OH | H | OH |
| Isourolithin A | H | H | OH | H | H | OH |
| Isourolithin B | H | H | OH | H | H | H |
| Urolithin M-5 | OH | OH | OH | OH | H | OH |
| Urolithin M-6 | H | OH | OH | OH | H | OH |
| Urolithin M-7 | H | OH | H | OH | H | OH |

In practice, for commercial scale products, it is convenient to synthesise the urolithins. Routes of synthesis are described, for example, in WO2014/004902, WO 2015/100213 and WO 2019/168972 (all of which are incorporated by reference).

Urolithins of any structure according to structure (I) may be used in compositions of the invention. Particularly suitable compounds for use in compositions of the invention are the naturally-occurring urolithins. Thus, Z is preferably OH and W, X and Y are preferably all H. When W, X and Y are all H, and A, and B are both H, and C, D and Z are all OH, then the compound is Urolithin C. When W, X and Y are all H, and A, B and C are all H, and D and Z are both OH, then the compound is Urolithin A. Preferably, the Urolithin used in a formulation of the invention is Urolithin A, Urolithin B, Urolithin C or Urolithin D. Most preferably, the Urolithin used in a formulation of the invention is Urolithin A.

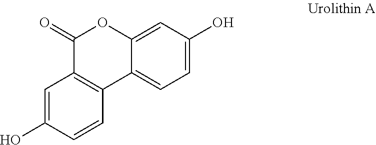 [SMILES: O=c1oc2cc(O)ccc2c2ccc(O)cc12]

In one embodiment, the urolithin for use in compositions of the invention is micronized. Micronization enables the urolithin to disperse in formulation or dissolve more rapidly. If micronized urolithin is used, then preferably, the urolithin has a D₅₀ size of under 8 μm-that is to say that 50% of the urolithin by mass has a particle diameter size of under 8 μm. More preferably, the urolithin has a D₅₀ size of under 7 μm, for example under 6 μm, for example under 5 μm, for example under 4 μm, for example under 2.5 μm. More preferably, the urolithin has a D₅₀ in the range 1.5 to 8.5 μm, for example 2 to 8 μm, for example 1.5 to 3.5 μm, for example 1.5 to 2.5 μm, for example 3 to 8 μm, for example 3 to 6 μm. Preferably, the urolithin has a D₉₀ size of under 25 μm. More preferably, the urolithin has a D₉₀ size of under 15 μm, for example under 12 μm. The urolithin preferably has a D₉₀ in the range 5 to 25 μm, for example 9 to 25 μm, for example 10 to 25 μm, for example 10 to 20 μm, for example 5 to 15 μm. Preferably, the urolithin has a D₁₀ in the range 0.5-3 μm, for example, 0.5 to 2.5 μm, for example, 0.5 to 2.0 μm, for example, 0.5 to 1.5 μm. Preferably, the urolithin has a D₉₀ in the range 5 to 25 μm, a D₅₀ in the range 1.5 to 8.5 μm and a D₁₀ in the range 0.5 to 3 μm.

Micronisation can be achieved by methods established in the art, for example compressive force milling, hamermilling, universal or pin milling, or jet milling (for example spiral jet milling or fluidised-bed jet milling) may be used. Jet milling is especially suitable.

### Forms of Compositions:

The uses and methods of the present invention preferably involve oral administration of compounds or compositions of the invention. Any suitable oral composition may be used. Accordingly, the use of a range of compositions which are suitable for oral administration, is envisaged. Thus, in some embodiments, the composition is administered in the form of an oral composition and one or more excipients suitable for oral administration. Oral compositions may comprise compositions having the form of a pill, tablet, capsule, caplet, lozenge, pastille, granules, powder for suspension, oral solution, oral suspension, oral emulsion, syrup, or the like. Compositions comprising compounds of formula (I) can be found in International Application, publication number: WO 2017/036992.

In a further embodiment of the invention, the composition is administered by any means known to the skilled person for administration such as, intramuscular, sublingual, cutaneous, inhalation and auricular. Oral administration is preferred.

Compositions may take any physical form suitable for the intended application, for example, they may be in the form of a solid (for example, a tablet or capsule), a semi-solid (for example, a softgel), or a liquid (including emulsions). In some instances, the composition may be in the form of a viscous fluid or a paste. Semi-solid forms may likewise contain excipients conventional in the art. The excipients can, for example, provide a desired hardness, shelf-life and flavour such that the composition has an acceptable taste, an attractive appearance and good storage stability. Semi-solid forms can be in the form of a paste. Where the composition is a softgel, it may for example be provided in a capsule having a shell. The shell may be of a conventional type, for example it may be a soft gelatin-based shell. By way of example, the composition may also be provided inside a hard capsule type of shell. Liquid compositions may be in the form of a medicine, a dietary supplement, or a beverage, each for oral consumption. Liquid formulations may be solutions, emulsions, slurries or other semi-liquids. Excipients in a liquid composition can, for example, provide a shelf-life, visual appearance, flavour and mouth-feel such that the composition has an acceptable taste, an attractive appearance and good storage stability. At certain levels of dilution, a drink may need to be shaken before the subject drinks it, so as to maintain an even suspension of the active ingredient.

In one embodiment of the invention, there is provided a composition of the invention comprising a pharmaceutically acceptable carrier.

### Additional Components in Compositions of the Invention:

Uses and methods of the invention comprise compounds of formula (I) and may comprise one of more additional components.

In one embodiment, the additional components comprise one of more components selected from an NAD+ booster, a mitochondrial booster, berberine, dehydroepiandrosterone, co-enzyme Q10, ubiquinol, evening primrose oil, omega 3 fatty acids, chasteberry (vitex), inositol, vitamin A, vitamin D, omega-3 fatty acids, pyrroloquinoline quinone, folic acid, salts of folic acid, carnitine, vitamin B12, alpha lipoic acid, iodine, zinc supplements and iron supplements. In a further embodiment, additional components comprise one of more components selected from trigonelline and oleuropein. These components may be of different levels of purity, including having an origin of natural extracts containing them.

In a further embodiment, the additional components comprise one of more components selected from: berberine, chaste berry, dehydroepiandrosterone, co-enzyme Q10, ubiquinol, evening primrose oil, omega 3 fatty acids, chasteberry (vitex), inositol, vitamin A, vitamin D, omega-3 fatty acids, pyrroloquinoline quinone, folic acid, salts of folic acid, carnitine, vitamin B12, alpha lipoic acid, iodine, zinc supplements and iron supplements.

In a further embodiment, the additional components comprise one of more vitamins or minerals. For example, the vitamin(s) comprise one or more of the following: Beta-Carotene, Biotin, Vitamin A (for example, vitamin A acetate), Vitamin B1 (for example, thiamine mononitrate), Vitamin B2 (riboflavin), Vitamin B3, Vitamin B6 (pyridoxine hydrochloride), Vitamin B12 (cyanocobalamin), Vitamin C (ascorbic acid), Vitamin D (cholecalciferol), Vitamin E (dl-α tocopheryl acetate), Folate (folic acid), Niacinamide and Pantothenic Acid (for example, calcium d-pantothenate). And/or, for example, wherein the mineral(s) is selected from one or more of the following: Calcium (for example, calcium carbonate). Chromium (for example, chromium chloride), Copper (for example, cupric citrate), Iodine (for example, potassium iodide), Iron (for example, ferrous fumarate), Magnesium (for example, magnesium oxide), Manganese (for example, manganese gluconate dihydrate), Molybdenum (for example, sodium molybdate), Selenium (for example, sodium selenite) and Zinc (for example, zinc oxide).

In a further embodiment, the additional components comprise one of more NAD+boosters. For examples, an NAD+booster is selected from: niacin, nicotinamide riboside, nicotinamide, nicotinic acid riboside, nicotinic acid mononucleotide, nicotinic acid adenine dinucleotide, nicotinamide mononucleotide, IHN, CD38 inhibitors (such as quercetin, luteolin, apigenin, 78c, luteolinidin and kuromanin), PARP inhibitors (such as BGB-290, olaparib, rucaparib, veliparib, CEP-9722, E7016, talazoparib, iniparib, niraparib, PJ34, DPQ and 3-aminobenzamide), SARM inhibitors (such as XAV939) and NAMPT activators (such as P7C3).

In a further embodiment, the additional components comprise one of more mitochondrial boosters. For example, a mitochondrial booster is selected from: resveratrol, pterostilbene, L-carnitine, berberine, epicatechin, epigallocatechin gallate (EGCG), curcumin, quercetin and Co-enzyme Q₁₀.

When a compound of formula (I) is administered directly to a subject, to modulate fertility. In general, compounds of formula (I) only have utility in increasing fertility when the subject is post-puberty. For example, a subject, such a female subject, such as a human female in the age range about 30 years to about 60 years, for example, about 35 to about 50 years, for example, about 35 to about 60 years.

### Additional Components in Culture Media of the Invention:

Cell culture media comprising compounds of formula (I) may also comprise one of more additional components. In a further embodiment, the one or more additional components are selected from one or more components list under ‘Additional components in compositions of the invention’ listed above.

### Dosing

The effective amounts of compounds of formula (I) for uses and methods of the invention will depend on whether compounds are being administered directly to subjects or whether the compounds are being added to cell culture media.

### Dosing (Directly to Subjects)

When administering directly to subjects, the effective amount of the composition to be taken will vary depending upon the manner of administration, the age, body weight, and general health of the subject. Factors such as the age, and weight of the subject may be important, and dosage regimens may be adjusted to provide the optimum response.

International patent applications WO 2018/162650 (Amazentis) and WO 2018/162651 (Amazentis) disclose optimised dose ranges for oral administration of urolithins.

The administration of compositions of the present invention preferably involves oral administration of a compound of formula (I) or salt thereof to a subject in a daily amount in the range of about 1.1 to about 8.8 mmol, for example, from about 1.7 to 6.0 mmol per day, for example, from about 1.7 to about 2.7 mmol per day, or from about 2.8 to about 6.6 mmol per day. As discussed below, administration is preferred in the range 100 mg to 2000 mg of a compound of Formula (1), for example, urolithin A, for example, 250 mg to 2000 mg urolithin

A (which corresponds to about 1.1 to 8.8 mmol), for example 250 mg to 1500 mg, such as 250 mg to 1000 mg, which results in a surprisingly good pharmacokinetic profile. In one embodiment the dose is 100 mg/day, in an alternative 125 mg/day, in an alternative 250 mg/day, in an alternative embodiment the dose is 500 mg/day and in another embodiment the dose is 1000 mg/day. In a further embodiment, the dose is 1500 mg/day. In a further embodiment, the dose is 2000 mg/day.

In a further embodiment, administration doses are selected from:
100 mg once or twice a day;
125 mg once or twice a day;
250 mg once or twice a day;
500 mg once or twice a day;
750 mg once or twice a day;
1000 mg once or twice a day;
1250 mg once or twice a day; or
1500 mg once or twice a day

The methods of the present disclosure would usually require daily administration of the compound of formula (I) or salt thereof, or of a composition containing the compound or salt, for a period over several months. In some embodiments, the methods may involve administration of the compound of formula (I), or salt thereof, over for example daily for at least 2 weeks, 3 weeks, 4 weeks, 5 weeks, 6 weeks, 8 weeks, 12 weeks, 4 months, 6 months, or for at least a year.

In a further embodiment a compound of formula (I) is administered to a male subject for about 2 months to about 4 months, for example for about 3 months.

In a further embodiment there is provided a compound of formula (I) for use in a method of improving seprmatogenesis in a male subject.

In a further embodiment there is provided a compound of formula (I) for use in a method of improving seprmatogenesis in a male subject, wherein the compounds of formula (I) is administered for about 2 months to about 4 months, for example for about 3 months.

The uses and methods of the present invention involve daily administration of the compound of formula (I) or salt thereof, or of a composition comprising the compound or salt. In some embodiments, the compound or composition is administered once per day, i.e. the compound or composition is to be administered at least once per 24 hour period. In other embodiments the compound, or composition comprising the compound, is administered multiple times per day, for example twice per day, or three or four times per day. In such cases, the daily dosage is divided between those multiple doses. In one embodiment administration is once a day, in a second embodiment administration is twice a day, in a third embodiment administration is three times a day.

The uses or methods of the present disclosure require daily administration of an amount of compound of formula (I) or salt thereof, of from 0.7 mmol per day up to 2.7 mmol per day thereof or from 0.7 mmol twice per day up to 2.7 mmol twice a day. In some embodiments, the amount administered is in the range of from 2.0 to 2.5 mmol. In some embodiments, the amount administered is approximately, 1.1, 1.2, 1.3, 1.4. 1.5, 1.6 1.7, 1.8, 1.9, 2.0, 2.1, 2.2, 2.3, 2.4, 2.5, 2.6, or 2.7 mmol. In some preferred embodiments the uses or method involves administration of approximately 2.2 mmol per day or 2.2 mmol twice per day of the compound of formula (I) or salt thereof (e.g. of urolithin A). The exact weight of compound that is administered depends on the molecular weight of the compound that is used. For example, urolithin A has a molecular weight of 228 g/mol (such that 2.20 mmol is 501.6 mg) and urolithin B has a molecular weight of 212 g/mol (such that 2.20 mmol is 466.4 mg).

In a further embodiment, the methods of the present disclosure require daily administration of an amount of compound of formula (I) or salt thereof, of from 2.8 mmol per day up to 6.0 mmol per day or twice per day thereof. In some embodiments, the amount administered is in the range of from 4.0 to 4.8 mmol. In some embodiments, the amount administered is approximately, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, or 6.0 mmol. In some preferred embodiments the use or method involves administration of approximately 4.4 mmol per day or twice per day of the compound of formula (I) or salt thereof (e.g. of urolithin A). The exact weight of compound that is administered depends on the molecular weight of the compound that is used. For example, urolithin A has a molecular weight of 228 g/mol (such that 4.40 mmol is 1003.2 mg) and urolithin B has a molecular weight of 212 g/mol (such that 4.40 mmol is 932.8 mg).

In some embodiments the methods involve administration of urolithin A in an amount in the range of from about 100 mg to about 600 mg/day for example, from about 125 mg to about 600 mg/day for example, about 200 mg to about 600 mg per day, for example from about 300 mg to about 600 mg/day, from 400 to 600 mg/day or an amount of urolithin A in said ranges twice per day. In a preferred embodiment the method involves administration of urolithin A in an amount in the range of from 450 to 550 mg, more preferably approximately 500 mg per day or twice per day. In a further preferred range, the method involves administration of urolithin A in the range from about 200 mg to about 300 mg, more preferably about 250 mg per day or twice per day. In a yet further preferred range, the method involves administration of urolithin A in the range from about 100 mg to about 150 mg, more preferably about 125 mg per day or twice per day.

In other embodiments the methods involve administration of urolithin A in an amount in the range of from 700 to 1300 mg/day twice per day, or in the range of from 750 to 1250 mg, or in the range of from 800 to 1200 mg, or in the range of from 850 to 1150 mg, or in the range of from 900 to 1100 mg per day or twice per day. In a preferred embodiment the method involves administration of urolithin A in an amount in the range of from 950 to 1150 mg/day or twice per day, more preferably approximately 1000 mg/day or twice per day.

In some preferred embodiments, the uses or methods involve administering urolithin A to the subject in an amount in the range of from 4.5 to 11 mg/kg/day, such as 4.5 to 8.5 mg/kg/day. In another embodiment, the uses or methods involve administering urolithin A to the subject in an amount in the range of 5 to 9 mg/kg/day. In another embodiment, the uses or methods involve administering urolithin A to the subject in an amount in the range of from 6.0 to 8 mg/kg/day.

In other preferred embodiments, the uses or methods involve administering urolithin A to the subject in an amount in the range of from 9 to 18 mg/kg/day such as 9 to 17 mg/kg/day. In another embodiment, the uses or methods involve administering urolithin A to the subject in an amount in the range of from 10 to 17 mg/kg/day. In another embodiment, the uses or methods involve administering urolithin A to the subject in an amount in the range of from 11 to 16 mg/kg/day.

Dosage regimes which combine two or more of a 125 mg, a 250 mg, a 500 mg dose and a 1000 mg dose may be advantageous. For example, a twice daily dosage regime which combines a first dose of 1000 mg and a second dose several hours later of 500 mg or a first dose of 500 mg and a second dose hours later of a 250 mg dose, or a 250 mg and a second dose hours later of a 125 mg dose. Said 125 mg, 250 mg or 500 mg dose may be 6-18 hours after the 250 mg, 500 mg or 1000 mg dose, for example 8-12 hours after the 250 mg, 500 mg or 1000 mg dose. For example, about 12 hours after the 250 mg, 500 mg or 1000 mg dose. Thus, according to a further aspect of the invention there is provided the treatment of a disease, disorder or condition with a compound of Formula (I) which comprises a twice daily dosage regime comprising a first dose of 250 mg or 500 mg or 1000 mg, followed by a second dose of 500 mg wherein the two doses are separated by 6-18 hours.

The compound of formula (I) or salt thereof, or composition containing the compound of salt, may be administered at any suitable time, for example, it may be administered in the morning after sleep or in the evening. In some embodiments, it may be preferable for the method to be performed at approximately the same time(s) each day, for example within 15, 30, 60 or 120 minutes of a given time point.

In a further embodiment, there is provided a composition wherein the compound of formula (I) is administered at a dose of about 4.5-18 mg/kg.

In a further embodiment, the compound of formula (I) is administered, to a subject, in the range about 50 mg to about 1000 mg

### Dosing (Cell Culture Media)

In a further embodiment, where a compound of formula (I) is added to cell culture medium the compound of formula (I) is added to the cell culture medium at a dose between about 1 μM to about 100 μM, for example, about 1 μM to about 50 μM.

In a further embodiment, where a compound of formula (I) is added to cell culture medium the compound of formula (I) is added to the cell culture medium at a dose between about 0.01 μM to about 10 μM, about 0.05 μM to about 10 μM, for example, about 0.1 μM to about 8 μM, about 0.1 μM to about 5 μM, about 0.2 μM to about 4 μM, about 0.5 μM to about 3 μM, about 0.5 μM to about 2 μM, about 0.1 μM to about 1 μM or about 0.05 μM to about 1 μM.

The term ‘about’ refers to a tolerance of +20% of the relevant value, for example ±15% of the relevant value, such as +10% of the relevant value or +5% of the relevant value.

The term ‘bovines’ includes cows, oxen, goats, sheep, bison, and buffalo.

The term ‘Pharmaceutically acceptable carrier’ means any carrier, diluent or excipient which is compatible with the other ingredients of the formulation and not deleterious to the recipient. The active agent may be formulated into dosage forms according to standard practices in the field of pharmaceutical preparations. See Alphonso Gennaro, ed., Remington's Pharmaceutical Sciences, 18th Edition (1990), Mack Publishing Co., Easton, Pa.

The term ‘ovarian reserve’ refers to the capacity of the ovary to provide oocytes that are capable of developing into eggs, capable of fertilization resulting in a healthy and successful pregnancy.

The term ‘puberty’ refers to the period during which adolescents reach sexual maturity and become capable of reproduction. On average, females begin puberty at ages 10-11 and complete puberty at ages 15-17; males generally begin puberty at ages 11-12 and complete puberty at ages 16-17.

### EXAMPLES

The following Examples illustrate the invention.

### Example 1

Young (4 months old) and middle-aged (12-14) months old female outbred mice are broken into two groups; n=22-24 control and n=22-24 test.

UA and control diet supplementation of test group females are for 8 weeks.

Tissue collections by dissection following cervical dislocation include:

Natural matings for confirmation of fertility by measuring fetal weight, length and morphology.

Ovarian sections for H&E staining and follicular characterization.

Oocytes and granulosa cells mitochondrial health are evaluated via the following readouts:
mtDNA copy number by qPCR.
mitochondrial membrane potential by JC-1 dye or MitoTracker CMXRos
ROS analysis by MitoSOX.
Gene expression analysis by qPCR of Drp1, Mff, Pink1, Parkin and Sod1

### Example 2

Sperm samples from five healthy men donors were processed within 1 hour of collection.

3 samples were either diluted 1:1 in culture media containing DMSO or urolithin A (UA) (0.05, 0.1, 0.5, and 2.0 μM in DMSO). For these samples, all treatments contained the same amount of DMSO. Two samples were pre-diluted in culture media, which was then diluted in the treatment solutions, immediately. This was to decrease the eventual effect of seminal plasma.

Samples were incubated for 4 hours with the indicated UA doses.

Results indicate that UA consistently increased multiple parameters associated with improved sperm quality. For instance, UA increased at all doses “Motile sperm concentration” expressed as millions of sperm cells per mL. Motile sperm concentration refers to the concentration of sperm in the sample with motility (rapid progressive, slow progressive, and non-progressive). It refers to the concentration (count/volume) of sperm within a sample that present any motility pattern. A higher number of motile sperm indicates better energy-producing pathways and higher chances of achieving fertilization (WHO laboratory manual for the examination and processing of human semen (6^{th} edition), https://www.who.int/publications/i/item/9789240030787). UA increase ‘motile sperm concentration from 2% at the lower dose of UA 0.05 μM to 21.4% at the highest UA 2 μM dose (Table 1).

UA also increased at all doses “Rapid progressive motile sperm concentration” expressed as millions of sperm cells per mL. Rapid progressive motile sperm concentration refers to the concentration of sperm in the sample with rapid progressive motility (>25 μm/s). It refers to the concentration (count/volume) of sperm within a sample that present a rapid progressive motility pattern. Rapidly progressive sperm numbers reflect sperm with the highest chance of achieving fertilization (WHO laboratory manual for the examination and processing of human semen (6^{th} edition), https://www.who.int/publications/i/item/9789240030787).

UA increased motile sperm concentration from 2% at the lower dose of UA 0.05 μM to 20% at the highest UA 2 μM dose (Table 1).

Finally, in the motile sperm, there was an increase by UA at all doses in sperm “Beat Cross-Frequency”. Beat cross frequency (BCF) is a parameter used in computer-assisted semen analysis (CASA) to evaluate sperm motility. It refers to the time-average rate at which the curvilinear path of a sperm crosses its average path trajectory (Sloter et al (2006) Human Reproduction, 21 (11), 2868-2875, https://doi.org/10.1093/humrep/del250). Essentially, it measures how frequently the sperm's head crosses its own average path, providing insight into the regularity and consistency of its movement. BCF is one of the parameters used to assess sperm quality and motility, which are important factors in determining male fertility (Larsen et al (2000) Human Reproduction 5 (7), 1562-1567, https://doi.org/10.1093/humrep/15.7.1562). UA increased BCF from 5% at the lower dose of UA 0.05 uM to 7% at the highest UA 2 uM dose (Table 1).

Overall, these data indicated that UA increases sperm motility and hence improved sperm health, when applied ex vivo to human sperm, and especially at a range of doses between 0.1 uM to 2 uM. This indicates higher semen quality and better energy-producing pathways that can potentiate higher fertilization rates.

TABLE 1

| | | Rapid progressive | |
|---|---|---|---|
| | Motile sperm | motile sperm | BCF Beat |
| | concentration | concentration | Cross- |
| | (million/mL) | (million/mL) | Frequency |
| 0.05 uM UA vs. | 2% | 2% | 5% |
| DMSO | | | |
| 0.1 uM UA vs. DMSO | 16.8% | 20% | 6% |
| 0.5 uM UA vs. DMSO | 10.7% | 10% | 9% |
| 2 uM UA vs. DMSO | 21.4% | 20% | 7% |

### Methods:

For the study, semen samples were obtained by masturbation after 2-7 days of ejaculatory abstinence, according to World Health Organization Guidelines (https://www.who.int/publications/i/item/9789240030787). Samples were collected into sterile wide-mouth polypropylene containers tested for sperm toxicity (BIRR semen collection container, IVF store code 113102). After collection, samples were kept in an air incubator for 1 hour, until semen liquefaction.

For experiments, a 50 mM stock Urolithin A (UA) solution was prepared in Dimethyl Sulfoxide (DMSO, Fisher Scientific, BP231-100). At the day of the experiments, a working solution was prepared by diluting the 50 mM stock solution 1:1000 in Sperm Washing Medium (FujiFilm Modified HTF Medium with Human Serum Albumin, 5.0 mg/mL, catalog ID: 9983). A blank working solution containing only DMSO was also prepared by diluting DMSO 1:1000 in Sperm Washing Medium. Treatment solutions were then prepared containing 0, 0.1 μM, 0.2 μM, 1.0 μM, and 4.0 μM were prepared as follows:

| | Sperm Washing | Working DMSO | Working UA |
|---|---|---|---|
| Treatment solution | Medium (μL) | solution (μL) | solution(μL) |
| 0 | 460.0 | 40.0 | 0.0 |
| 0.1 μM | 460.0 | 39.0 | 1.0 |
| 0.2 μM | 460.0 | 38.0 | 1.0 |
| 1.0 μM | 460.0 | 30.0 | 10.0 |
| 4.0 μM | 460.0 | 0.0 | 40.0 |

Treatment solutions were then diluted 1:1 with raw semen (samples A, B, and D) or with semen that had been pre-diluted 1:1 in Sperm Washing Medium (samples C and E), so that final achieved treatment concentrations were 0 (blank, DMSO only, 0.05 UM, 0.1 μM, 0.5 μM, and 2.0 UM UA. This was incubated at 36° C. for 4 hours, after which motility was assessed using a CEROS II system for computer-assisted semen analysis (Hamilton Thorne Inc., Beverly, MA, USA). To achieve this, 4-chamber 20 μm Leja slides (IMV International Ref. 025107-025108) were pre-heated at 37° C. 2.5 μL of each sample was pipetted into each chamber, and the Human Motility module of the CEROS II system was used to capture multiple fields (at least 6) in order to determine sperm motility parameters.

### Example 3

### A Randomized, Placebo-Controlled Trial to Show the Effect of Urolithin a Supplementation on Boosting Semen Health to Reverse Male Infertility

A total of n=120, adult males (18+ years of age) with varicocele are recruited into the clinical study and randomized into either a placebo (n=40); 500 mg Urolithin A intervention (n=40) or a 1000 mg Urolithin A intervention (n=40). The length of intervention is 90 days. Semen samples are collected prior to the start of the study, at the intermediate point of the study (Day 45) and end of study visit (Day 90).

Inclusion and exclusion criteria for the recruitment are the following:
Subjects with medical history of infertility for at least 12 months.
No history of surgical or medical treatments that were performed for infertility
Varicocele was diagnosed with ultrasonography and graded as grade I, palpable only with the Valsalva maneuver; Grade II, palpable without the Valsalva maneuver; Grade III, visible from a distance.

The primary outcome of the study is on semen health (defined as):
Sperm count
Sperm motility and morphology (Who-6 index)

Key secondary outcomes include:
Serum concentrations of male (testosterone) hormone
oxidative stress and inflammatory biomarkers
mitochondrial health parameters on sperms

### Example 4

### A Randomized, Placebo-Controlled Trial to Show the Effect of Urolithin a Supplementation on Boosting Ovarian Reserve to Boost Female Infertility

A total of n=120, adult females (18-45 years of age) with failure to conceive in last 12 months are recruited into the clinical study and randomized into either a placebo (n=30); 500 mg Urolithin A intervention (n=30) or a 1000 mg Urolithin A intervention. The length of intervention is n=90 days. Study endpoints are collected prior to the start of the study, at the intermediate point of the study (Day 45) and end of study visit (Day 90).

Inclusion and exclusion criteria for the recruitment are the following:
Subjects with medical history of infertility for at least 12 months.
No history of surgical or medical treatments that were performed for infertility

The primary outcome of the study is:

Improved egg quality, increased/improved egg function, increase in egg number, percentage of normal eggs vs abnormal eggs, ability to fertilize and improvement of implantation Measurements of ovarian reserve as defined via both a) Blood test (AMH, E2, FSH) and b) Ultrasound measurement of antral follicle count and ovarian volume

Key secondary outcomes include:
Plasma levels of sex hormone binding globulin (SHBG), morning cortisol, insulin growth factor 1 (IGF-1), testosterone, dehydroepiandrosterone, follicle stimulating hormone (FSH), and estradiol.

1. A method of enhancing or improving fertility, comprising administering to a male subject or a female subject in need thereof an effective amount of a compound of formula (I), or a salt thereof, 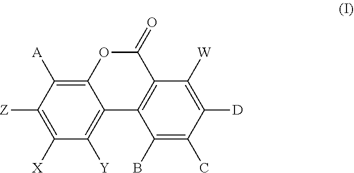
wherein:
A, B, C, D, W, X, Y and Z are each independently selected from H and OH.

2. The method of claim 1, wherein the subject is a male subject.

3. The method of claim 2, wherein the enhancement or improvement in fertility further comprises increasing pregnancy success rate or extending reproductive longevity in a male subject.

4. (canceled)

5. The method of claim 2, wherein the enhancement or improvement in fertility in the male subject comprises:
a. an improvement in sperm quality;
b. an increase in sperm quantity;
c. an improvement in sperm motility;
d. an increase in rapid progressive motile sperm quantity;
e. an increase in sperm beat cross frequency;
f. an improvement in sperm maturation;
g. an improvement in sperm vitality;
h. an improvement in sperm function; and/or
i. an improvement in sperm health.

6. (canceled)

7. The method of claim 1, wherein the enhancement or improvement in fertility comprises treatment of prophylaxis of a disease, condition or disorder causing male infertility, wherein the disease, condition or disorder is selected from oligozoospermia, azoospermia, teratospermia and varicocele.

8. (canceled)

9. The method of claim 2, wherein the enhancement or improvement in fertility in the male subject, comprises one of more of the following:
a. an enhancement of sperm mitochondrial function;
b. an increase in sperm mitochondrial mass;
c. an enhancement of sperm quality; and/or
d. an increase in the quantity of mitochondria in sperm.

10. The method of claim 1 wherein the subject is a female subject.

11. The method of claim 10, wherein the enhancement or improvement in fertility comprises maintaining or enhancing ovarian health.

12. The method of claim 11 wherein the enhancement or improvement in fertility comprises enhancing of ovarian health.

13. The method of claim 11 wherein the enhancing of ovarian health comprises (i) maintaining or enhancing ovarian reserve in a female subject; or (ii) treating polycystic ovary syndrome or pre-eclampsia.

14. (canceled)

15. A method of increasing oocyte mitochondrial mass; preventing or slowing a decrease of oocyte mitochondrial mass; delaying perimenopause, menopause, or a symptom thereof; or egg harvesting, comprising administering to a female subject in need thereof an effective amount of a compound of formula (I), or a salt thereof, 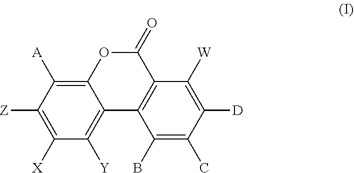
wherein:
A, B, C, D, W, X, Y and Z are each independently selected from H and OH.

16. (canceled)

17. (canceled)

18. The method of claim 1, wherein the compound of formula (I) is administered in the range about 50 mg/day to about 2000 mg/day.

19. The method of claim 1, wherein the compound of formula (I) is urolithin A, urolithin B, urolithin C or urolithin D, or a combination of any of them.

20. A method of human in-vitro fertilization, comprising administering to a female subject in need thereof an effective amount of a compound of formula (I), or salt thereof; or of delaying aging of an egg cultured in vitro, comprising injecting the compound of formula (I), or a salt thereof, directly into the egg, 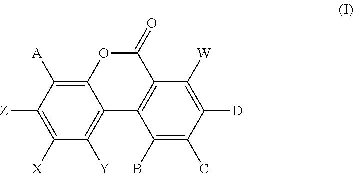
wherein:
A, B, C, D, W, X, Y and Z are each independently selected from H and OH.

21. The method of claim 20, comprising improving egg fertilization rate or improving embryo development during in-vitro fertilization.

22. (canceled)

23. (canceled)

24. A cell culture medium for use in in vitro fertilization which comprises a compound of formula (I), or a salt thereof, 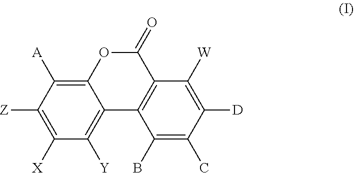
wherein:
A, B, C, D, W, X, Y and Z are each independently selected from H and OH.

25. (canceled)

26. The method of claim 20, wherein the compound of formula (I) is administered to the subject after the fertilized egg or embryo has been transferred to the subject.

27. (canceled)

28. (canceled)

29. The method of claim 1, wherein the subject is a human female in the age range of about 30 years to about 60 years.

30. The method of claim 1, further comprising administering one or more additional components, vitamins, or minerals,
wherein the one or more additional components are selected from an NAD+ booster, a mitochondrial booster, berberine, dehydroepiandrosterone, co-enzyme Q10, ubiquinol, evening primrose oil, omega 3 fatty acids, chasteberry (vitex), inositol, vitamin A, vitamin D, omega-3 fatty acids, pyrroloquinoline quinone, folic acid, salts of folic acid, carnitine, vitamin B12, alpha lipoic acid, iodine, zinc supplements, iron supplements, trigonelline and oleuropein;
the one or more vitamin(s) comprise one or more of the following: Beta-Carotene, Biotin, Vitamin A (for example, vitamin A acetate), Vitamin B1 (for example, thiamine mononitrate), Vitamin B2 (riboflavin), Vitamin B6 (pyridoxine hydrochloride), Vitamin B12 (cyanocobalamin), Vitamin C (ascorbic acid), Vitamin D (cholecalciferol), Vitamin E (dl-α tocopheryl acetate), Folate (folic acid), Niacinamide and Pantothenic Acid (for example, calcium d-pantothenate); and
the one or more mineral(s) comprise one or more of the following: Calcium (for example, calcium carbonate). Chromium (for example, chromium chloride), Copper (for example, cupric citrate), Iodine (for example, potassium iodide), Iron (for example, ferrous fumarate), Magnesium (for example, magnesium oxide), Manganese (for example, manganese gluconate dihydrate), Molybdenum (for example, sodium molybdate), Selenium (for example, sodium selenite) and Zinc (for example, zinc oxide).

31. (canceled)

32. (canceled)

33. (canceled)

34. (canceled)

35. (canceled)

36. The method of claim 1, wherein the compound of formula (I) is urolithin A.
